(19) United States
(12) Patent Application Publication
(10) Pub. No.: US 2025/0349010 A1
(43) Pub. Date: Nov. 13, 2025

(54) FLOW MEASUREMENT WITH DUAL ENERGY CT

(71) Applicant: The Regents of the University of California, Oakland, CA (US)

(72) Inventor: Sabee MOLLOI, Oakland, CA (US)

(73) Assignee: The Regents of the University of California, Oakland, CA (US)

(21) Appl. No.: 19/204,245

(22) Filed: May 9, 2025

Related U.S. Application Data
(60) Provisional application No. 63/644,998, filed on May 9, 2024.

(51) Int. Cl. G06T 7/00; A61B 6/00; A61B 6/03; A61B 6/50; G06T 7/12; G06T 7/136; G06T 7/246; G16H 40/63; G16H 50/30

(52) U.S. Cl. CPC G06T 7/0016; A61B 6/032; A61B 6/481; A61B 6/504; G06T 7/12; G06T 7/136; G06T 7/246; G16H 40/63; G16H 50/30; G06T 2207/10024; G06T 2207/10081; G06T 2207/30104

(57) ABSTRACT

A method for measuring blood flow in an organ of a subject includes a step of administering an iodine-based contrast agent to the subject and acquiring preparatory computed tomography (CT) images of the organ, referred to as preparatory CT images. The distribution of the iodine-based contrast agent is then monitored using bolus tracking based on the preparatory CT images and the contrast injection duration to determine a time of maximum contrast enhancement. At or near this identified time, dual-energy computed tomography (CT) images of the organ are acquired, referred to as dual-energy CT images. A curve fitting function is applied to the bolus tracking and dual-energy CT images to calculate iodine concentration over time. Based on the calculated iodine concentration, the blood flow rate in the organ is quantified. A system implementing the method is also provided.

### CROSS-REFERENCE TO RELATED APPLICATIONS

This application claims the benefit of U.S. provisional application Ser. No. 63/644,998 filed May 9, 2024, the disclosure of which is hereby incorporated in its entirety by reference herein.

### TECHNICAL FIELD

In at least one aspect, the present invention relates to medical imaging, medical physics, radiology, and cardiology. In particular, the present invention related to flow measurement with dual energy CT for different organs such as heart, lungs, brain, kidneys, liver, and the lymphatic system.

### BACKGROUND

There is a need for functional assessment of disease such as flow in different organs. For example, ischemic coronary heart disease is the world's leading cause of mortality and morbidity. Within this complex disease entity, many patients suffer from myocardial ischemia but are found to have no obstructed coronary arteries (INOCA). These patients have an elevated risk of cardiovascular events. Yet current methods for accurately diagnosing and assessing the physiological effects of INOCA are limited. Catheter-based approaches are invasive, with added risk, procedural time, and cost. Positron emission tomography (PET) and cardiac magnetic resonance (CMR), both noninvasive techniques for clinically assessing INOCA, have limitations such as claustrophobia (CMR), cost and radiation dose (PET), and local expertise and availability (both). None of these noninvasive tests accurately yields both anatomical information on the extent of coronary atherosclerosis and its pathophysiological consequences. There are low-dose dynamic CT perfusion techniques that can accurately measure myocardial perfusion in mL/min/g. However, these techniques are limited by patient motion misregistration artifacts. There are existing dual energy CT techniques that measure relative blood volume within an organ, but these techniques cannot be used to measure absolute flow in mL/min/g, which is necessary disease assessment in a population.

A known method for flow measurement using computed tomography (CT) involves the acquisition of two separate volume images-one before and one after the administration of a contrast agent. This approach requires image registration to align the two scans, which introduces significant susceptibility to motion artifacts, particularly under stress conditions where patient movement is common. These artifacts can impair image fidelity and limit the reliability of quantitative analysis. In this prior art, perfusion is estimated using first-order approximations, based on the time interval between the two volume acquisitions and the average iodine concentration measured in each scan. However, the method relies on Hounsfield Unit measurements rather than directly quantifying iodine concentration, thereby precluding accurate and standardized perfusion metrics in units such as milliliters per minute per gram (mL/min/g). The technique is further limited by its dependence on high-performance, ultra-fast CT scanners capable of capturing dynamic sequences rapidly enough for cardiac imaging applications. This constraint restricts broader clinical adoption. Moreover, the method requires manual segmentation of anatomical regions, which is time-consuming, subject to inter-user variability, and impractical for routine clinical workflows.

Although bolus tracking images and dual-energy acquisitions were used in some instances, the processing still involved averaging iodine concentrations over time intervals and lacked automation in segmentation and analysis. Consequently, these limitations-motion sensitivity, imprecise quantification, equipment demands, and manual workflow-significantly reduce the scalability and diagnostic utility of the method in typical clinical environments.

Accordingly, there is a need for improved techniques for measuring flow in organs.

### SUMMARY

In at least one aspect, dual-energy CT is used for flow measurement for different organs such as the heart, lungs, brain, kidneys, liver, and lymphatic system. The measurement can be done following a standard contrast injection similar to CT angiography. The bolus tracking images, along with the CT angiogram, can be used to measure flow. The flow measurement can be used for accurate functional assessment of disease in different organs.

In another aspect, a method for a flow measurement using dual-energy computed tomography (CT) is provided. This method allows for the assessment of blood flow in various organs such as the heart, lungs, brain, kidneys, liver, and lymphatic system following a standard contrast injection, similar to CT angiography. The technology utilizes bolus tracking images combined with a CT angiogram to measure flow, providing a functional assessment of disease in these organs.

In another aspect, the method improves upon the prior art by addressing problems associated with motion misregistration artifacts and the need for manual image segmentation, common in other flow measurement methods. The method can utilize a curve fitting function (e.g., a gamma variate function) for curve fitting to calculate iodine concentration and time for flow measurement. This approach is notable for its automated segmentation based on dual-energy material decomposition, making it more practical for routine clinical use and adaptable to all scanners capable of dual-energy CT.

In another aspect, a method for measuring blood flow in an organ of a subject is provided. The method includes a step of administering an iodine-based contrast agent to the subject and acquiring preparatory computed tomography (CT) images of the organ, referred to as preparatory CT images. The distribution of the iodine-based contrast agent is then monitored using bolus tracking based on the preparatory CT images to determine a time of maximum contrast enhancement. At or near this identified time, dual-energy computed tomography (CT) images of the organ are acquired, referred to as dual-energy CT images. A curve fitting function is applied to the dual-energy CT images to calculate iodine concentration over time. Based on the calculated iodine concentration, the blood flow rate in the organ is quantified and optionally expressed in milliliters per minute per gram (mL/min/g).

In another aspect, a system for measuring blood flow in an organ of a subject is provided. The system comprises a CT scanner capable of dual-energy acquisition configured to acquire images of the organ after administration of contrast agent (e.g., iodine based), and a contrast agent administration unit for delivering the contrast agent. The system further includes an image processing unit configured to determine a time of maximum contrast enhancement for preparatory CT images, receive dual-energy CT images of the organ, perform automated segmentation of the organ using dual-energy material decomposition, calculate iodine concentration using a curve fitting function, and quantify a blood flow rate in the organ based on the calculated iodine concentration.

In another aspect, a system for measuring blood flow in an organ of a subject is provided. The system comprises a dual-energy computed tomography (CT) scanner configured to acquire images of the organ following the administration of an iodine-based contrast agent, and a contrast agent administration unit for delivering the contrast agent. The system further includes an image processing unit configured to:
optionally control the administration of an iodine-based contrast agent to the subject;
acquire preparatory computed tomography (CT) images of the organ, referred to as preparatory CT images;
monitor distribution of the iodine-based contrast agent using bolus tracking based on the preparatory CT images;
determine a time of maximum contrast enhancement from the preparatory CT images and contrast injection duration;
acquire dual-energy computed tomography (CT) images of the organ at or near the determined time of maximum contrast enhancement, referred to as dual-energy CT images;
calculate iodine concentration over time using a curve fitting function based on the bolus tracking and dual-energy CT images; and
quantify a blood flow rate in the organ based on the calculated iodine concentration.

In another aspect, the dual-energy CT images are acquired following standard CT angiography contrast injection protocols.

In another aspect, the iodine concentration is determined by generating an iodine map from dual-energy CT data using material decomposition.

In another aspect, bolus tracking is performed by monitoring attenuation in an arterial input to detect contrast arrival.

In another aspect, utilization of dual energy CT reduces or eliminates motion misrepresentation artifacts which plague the prior art.

In another aspect, the invention provides a method for measuring blood flow in an organ of a subject by administering a contrast agent, acquiring dual-energy computed tomography (CT) images, utilizing bolus tracking to monitor the contrast agent, analyzing the images to determine the time of maximum enhancement, and calculating the flow rate based on iodine concentration using a curve fitting function (e.g., a gamma variate function).

In another aspect, the method is specified for use with particular organs, including the heart, lungs, brain, kidneys, liver, and the lymphatic system, demonstrating its versatility across different clinical applications.

In another aspect, the method employs an iodine-based contrast medium, which is a common type of contrast agent used in CT imaging, ensuring compatibility with existing medical practices and protocols.

In another aspect, an automated segmentation feature is incorporated into the method, where the organ is automatically distinguished from surrounding tissues in the dual energy CT images through material decomposition, enhancing the accuracy and efficiency of the process.

In another aspect, the measurement of the flow rate is explicitly quantified in terms of milliliters per minute per gram of tissue (mL/min/g), providing a standardized unit of measurement that can be universally applied for clinical assessments.

In another aspect, a system is introduced for measuring flow in an organ, which includes a dual energy CT scanner, a contrast agent administration unit, and an image processing unit equipped to manage various tasks from image acquisition to flow rate calculation, offering a comprehensive and integrated solution.

In another aspect, the system's image processing unit includes a segmentation module specifically designed to automate the segmentation of the organ based on dual energy material decomposition, further refining the system's capability in delivering precise and reliable results.

In another aspect, the integration of the CT scanner and the image processing unit into a single device is proposed, emphasizing the invention's emphasis on system efficiency and user-friendliness.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

For a further understanding of the nature, objects, and advantages of the present disclosure, reference should be had to the following detailed description, read in conjunction with the following drawings, wherein like reference numerals denote like elements and wherein:

FIG. 1. Schematic of a system for measuring blood flow in an organ of a subject using dual-energy computed tomography.

FIG. 2. Flow chart of a method for measuring blood flow in an organ of a subject using dual-energy computed tomography.

FIG. 3. Arterial input function for a two-volume CT perfusion technique where a non-contrast volume scan (V1) is acquired followed by contrast injection, bolus tracking (circles) with pulmonary artery triggering at 80 HU above the blood pool enhancement (black square), followed by acquisition of CT angiogram (V2) at approximately peak enhancement (white square).

FIGS. 4a and 4b. Linear regression (a) and Bland-Altman analysis (b) for lung mass associated with perfusion defect (MREF) and the MCP assigned lung mass distal to the balloon (MMCP).

FIGS. 5a and 5b. Linear regression (a) and Bland-Altman analysis (b) for percentage of perfusion defect mass and mass at risk based on MCP assignment.

FIG. 6. This figure shows the region of interest (black circle) within the pulmonary artery (top) and the associated gamma variate used to determine the actual peak contrast enhancement (bottom).

FIGS. 7a, 7b, 7c, and 7d. Representative flow map images from axial and coronal view of a normal (a and b) and a patient with emphysema (c and d) showing significant reduction of blood flow.

FIG. 8. Arterial input function for a patient with emphysema.

### DETAILED DESCRIPTION

Reference will now be made in detail to presently preferred embodiments and methods of the present invention, which constitute the best modes of practicing the invention presently known to the inventors. The Figures are not necessarily to scale. However, it is to be understood that the disclosed embodiments are merely exemplary of the invention that may be embodied in various and alternative forms. Therefore, specific details disclosed herein are not to be interpreted as limiting, but merely as a representative basis for any aspect of the invention and/or as a representative basis for teaching one skilled in the art to variously employ the present invention.

It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

With respect to the terms “comprising,” “consisting of,” and “consisting essentially of,” where one of these three terms is used herein, the presently disclosed and claimed subject matter can include the use of either of the other two terms.

It should also be appreciated that integer ranges explicitly include all intervening integers. For example, the integer range 1-10 explicitly includes 1, 2, 3, 4, 5, 6, 7, 8, 9, and 10. Similarly, the range 1 to 100 includes 1, 2, 3, 4 . . . 97, 98, 99, 100. Similarly, when any range is called for, intervening numbers that are increments of the difference between the upper limit and the lower limit divided by 10 can be taken as alternative upper or lower limits. For example, if the range is 1.1. to 2.1 the following numbers 1.2, 1.3, 1.4, 1.5, 1.6, 1.7, 1.8, 1.9, and 2.0 can be selected as lower or upper limits.

When referring to a numerical quantity, in a refinement, the term “less than” includes a lower non-included limit that is 5 percent of the number indicated after “less than.” A lower non-includes limit means that the numerical quantity being described is greater than the value indicated as a lower non-included limited. For example, “less than 20” includes a lower non-included limit of 1 in a refinement. Therefore, this refinement of “less than 20” includes a range between 1 and 20. In another refinement, the term “less than” includes a lower non-included limit that is, in increasing order of preference, 20 percent, 10 percent, 5 percent, 1 percent, or 0 percent of the number indicated after “less than.”

The term “one or more” means “at least one” and the term “at least one” means “one or more.” The terms “one or more” and “at least one” include “plurality” as a subset.

The term “substantially,” “generally,” or “about” may be used herein to describe disclosed or claimed embodiments. The term “substantially” may modify a value or relative characteristic disclosed or claimed in the present disclosure. In such instances, “substantially” may signify that the value or relative characteristic it modifies is within +0%, 0.1%, 0.5%, 1%, 2%, 3%, 4%, 5% or 10%.

The term “Dual-energy CT” refers to a computed tomography imaging technique that simultaneously or sequentially acquires CT images at two distinct X-ray energy levels. By utilizing differences in attenuation of X-rays at these two energy levels, dual-energy CT enables material decomposition, distinguishing between different tissue types or contrast materials based on their unique attenuation characteristics. This technology enhances diagnostic accuracy by providing quantitative and qualitative analyses that cannot be obtained with conventional single-energy CT imaging.

### Abbreviations

“AIF” means arterial input function.

“CMR” means cardiac magnetic resonance.

“CT” means computed tomography.

“HU” means Hounsfield units.

“INOCA” means ischemia with no obstructed coronary arteries.

“PET” means positron emission tomography.

In at least one aspect, the invention employs a dual-energy CT image acquired at peak contrast enhancement, informed by real-time bolus tracking data acquired at the aorta using thin-slice, high-speed scanning. In this context, peak contrast enhancement in the context of medical imaging (particularly in computed tomography (CT) using a contrast agent) refers to the point in time when the concentration of the contrast agent (such as iodine) within a specific anatomical region (like an artery, organ, or lesion) reaches its maximum value after injection. The bolus tracking images are sampled at a predetermined frame rate (e.g., 2-4 frames per second) until a predefined contrast arrival threshold is met. Once detected, a dual-energy CT image is acquired after a predetermined time interval (e.g., about 4 to 8 seconds). For example, the scanner performs real-time, low-dose imaging of a region such as the aorta while the iodine contrast agent circulates through the bloodstream. As each image is captured, it measures brightness or attenuation in Hounsfield Units (HU), which increases in proportion to the rising iodine concentration. A predefined HU value-often set at 100 HU above the baseline-is established as the arrival threshold. Once this threshold is reached, indicating contrast bolus arrival, the system automatically triggers the main dual-energy CT scan after a predetermined time interval (e.g., about 4 to 8 seconds). The arterial input function (AIF) is derived from the bolus tracking curve using a curve fitting function (e.g., a gamma variate function), while the iodine concentration in the target organ is extracted from the dual-energy image. Flow (Q) is then computed using the relation Q=ΔV/ΔT, where ΔV is the change in blood volume and ΔT is the time inferred from the AIF curve.

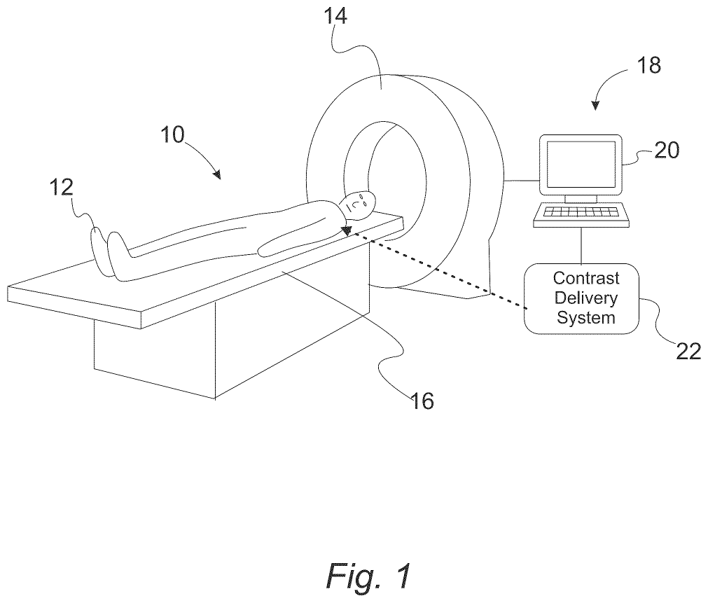

Referring to FIG. 1, a schematic illustrating an exemplary system 10 for measuring blood flow in an organ of a subject 12, according to aspects of the invention. The system includes a dual-energy computed tomography (CT) scanner 14 configured to acquire dual-energy CT images of the subject 12 positioned on a patient table 16. In a refinement, the dual-energy CT scanner 14 acquires data at two different energy levels (e.g., a first energy level from 80 kVp to 120 kVp and a second energy level from 130 kVp to 140 kVp). In a refinement, a contrast agent administration unit 22 is operably connected to the subject 12 via an intravenous delivery line (not labeled), enabling injection of an iodine-based contrast agent. The scanner 10 is communicatively coupled to an image processing unit 18, which may be a separate computer or integrated within the scanner. The image processing unit 18 is configured to implement the image manipulation steps of the method set forth below. The image processing unit 18 is configured to receive the dual-energy CT images, perform automated segmentation of the organ based on dual-energy material decomposition, determine a time of maximum contrast enhancement using bolus tracking, calculate iodine concentration using a curve fitting function (e.g., a gamma variate function), and quantify the blood flow rate in the organ, expressed in milliliters per minute per gram (mL/min/g). In particular, the image process unit 18 is configured to generate an arterial input function based on attenuation measurements from bolus tracking images. In a refinement, the image processing unit 18 is configured to output a perfusion map color-coded by blood flow values in milliliters per minute per gram (mL/min/g). As set forth below, the blood flow rate is quantified by integrating the iodine concentration over a segmented volume of the organ and dividing by a transit time extracted from the curve fitting function. In a further refinement, the image processing unit is further configured to apply a curve fitting function (e.g., gamma variate curve fit) to a time-attenuation profile generated from bolus tracking data as set forth below.

Still referring to FIG. 1, a display monitor 20 is operatively connected to image processing unit 18 to visualize images and calculated flow metrics. The contrast agent administration unit 22 may be operated via a manual or automated control interface (not shown) and is optionally integrated into the workflow of the image processing unit 18. Automated contrast agent administration units, also known as power injectors or automated injectors, are electromechanical devices designed to precisely control the volume, rate, and timing of contrast agent injection. These units deliver contrast synchronously with the imaging protocol, often using bolus tracking to optimize timing. Many systems support dual-barrel configurations that enable sequential injection of contrast agent followed by a saline flush, all according to a programmable sequence tailored to the specific imaging requirements. In a refinement, the contrast agent administration unit is an automated injector configured to deliver a bolus of iodine-based contrast followed by a saline flush. The system may be applied to a variety of organs, including the heart, lungs, brain, kidneys, liver, and lymphatic system.

In another aspect, bolus tracking image data is applied along with the dual energy image acquired at maximum enhancement. Bolus tracking is a real-time imaging technique used in CT to monitor the arrival and progression of a contrast agent (typically iodine-based) through the bloodstream and to automatically trigger image acquisition at the optimal moment-usually when the contrast reaches its peak concentration in a target vessel or organ. A curve fitting function can be applied for curve fitting. In certain refinements, a gamma variate function is used to fit the time-attenuation curve derived from bolus tracking. In alternative refinements, other models may be used, including exponential, log-normal, compartmental, or deconvolution-based methods, depending on system requirements and clinical protocols. A gamma variate function is a mathematical model commonly used to describe the time-concentration curve of a contrast agent in blood flow studies, particularly in medical imaging techniques like CT, MRI, or nuclear medicine. It provides a realistic, smooth approximation of how a bolus of contrast agent passes through the vasculature over time, helping to extract physiological parameters such as blood flow, volume, and mean transit time. The gamma variate function typically takes the form:

$$C ( t ) = A \cdot {( t - t 0 )}^{α} \cdot {e}^{- \frac{t - t 0}{β}} , t > t 0$$

wherein:
C(t) is the concentration of contrast agent at time t;
A is a scaling constant;
t₀ is the arrival time of contrast agent;
α is a shape parameter; and
β is a decay parameter (related to washout rate).

The gamma variate curve closely resembles the physiological response of a vascular system to a contrast bolus and is ideal for characterizing arterial input functions (AIF) in perfusion imaging. The rising portion of the curve models the inflow of contrast into the vessel or tissue, while the peak corresponds to the maximum concentration-often used as the triggering point for scanning. The falling portion represents the washout phase, during which the contrast agent exits the region. Therefore, this information is used to calculate the iodine concentration and time for flow measurement. This is in contrast to a prior art method that used the time difference between the two volume scans and the average iodine concentration in these two images, which are only first-order approximations. This prior art suffers from motion misrepresentation artifacts, which can be a major problem for most patients. The present embodiment uses dual-energy CT, which addresses this problem. Advantageously, the present method can utilize all scanners capable of dual-energy CT.

In another aspect, the curve fitting function is applied to a time-intensity curve generated from bolus tracking images acquired at a frame rate from 1 to 5 frames per second. In a refinement, the frame rate is from 3 to 4 frames per second.

In the context of the present invention, bolus tracking is employed as an integral component of dual-energy computed tomography (CT) to enhance the precision and reliability of blood flow measurements within various organs. Initially, a bolus of iodine-based contrast agent is intravenously administered to a patient. Subsequently, the dual-energy CT scanner performs continuous, real-time, low-dose imaging at a predetermined anatomical location, typically a major vessel such as the pulmonary artery or aorta, to monitor the passage of the contrast bolus. Specialized software tracks the contrast concentration by measuring enhancement in Hounsfield units (HU) at this anatomical location. Once the enhancement reaches a pre-specified threshold indicative of contrast bolus arrival, the software automatically triggers the primary dual-energy CT acquisition after a predetermined time interval (e.g., about 4 to 8 seconds). This optimally timed scan ensures maximal contrast enhancement, enabling precise differentiation and quantification of iodine concentration within tissues via dual-energy material decomposition. By capturing dual-energy CT images at this carefully determined time point, the invention allows for accurate calculation of absolute blood flow (expressed as milliliters per minute per gram, mL/min/g) utilizing a gamma variate function for curve fitting of the bolus tracking data. The integration of bolus tracking with dual-energy CT significantly reduces potential motion artifacts and improves clinical workflow by automating image segmentation, thereby providing simultaneous anatomical and functional assessments with enhanced accuracy and reduced radiation exposure and contrast dosage.

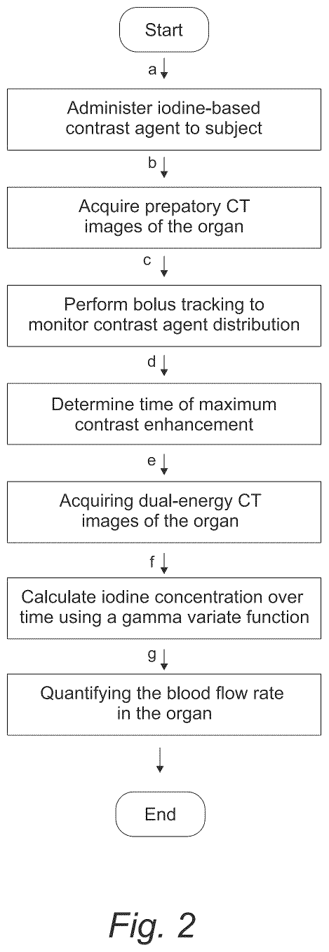

Referring to FIG. 2, a flowchart depicting a method for quantifying blood flow in an organ of a subject using the system of FIG. 1 is provided. In step a), an iodine-based contrast agent is administered to the subject. In step b), preparatory computed tomography (CT) images of the organ are acquired. These images are referred to as preparatory CT images and can be obtained by CT angiography contrast injection protocols. In step c), the distribution of the contrast agent is monitored using bolus tracking based on the preparatory CT images. In step d), an optimal imaging time point corresponding to maximum contrast enhancement is identified from the preparatory CT images and contrast injection time interval. In a refinement, the time of maximum contrast enhancement is half of the contrast injection duration (e.g. 2-4 seconds) and an organ-specific dispersion constant (e.g. 1-3 seconds). In step e), at or near this identified time, dual-energy computed tomography (CT) images of the organ at or near the determined time of maximum contrast enhancement are acquired. These images are referred to as dual-energy CT images. In step f), iodine concentration over time is calculated using a curve fitting function (e.g., a gamma variate function) based on the bolus tracking and the dual-energy CT images. In step g), the blood flow rate in the organ is quantified based on the calculated iodine concentration, expressed in grams per milliliter (g/mL). In a refinement, the iodine concentration is calculated in units of milligrams per milliliter and converted to blood volume using a calibration factor based on iodine density.

In another aspect, the blood flow rate in an organ is quantified based on calculated iodine concentration derived from bolus tracking and dual-energy computed tomography (CT) images, and the result is expressed in standardized physiological units of milliliters per minute per gram (mL/min/g). This process begins with the extraction of iodine concentration values from the dual-energy CT image acquired at or near peak contrast enhancement, using material decomposition techniques to generate iodine maps. In this context, material decomposition refers to a computational technique used in dual-energy computed tomography (CT) imaging, wherein image data acquired at two distinct X-ray energy levels is processed to distinguish and quantify the relative contributions of two or more basis materials—such as iodine, soft tissue, water, or calcium—within each voxel. The technique utilizes energy-dependent attenuation differences to generate material-specific images, including iodine maps, virtual non-contrast images, and effective atomic number or electron density maps. Material decomposition may be performed in the projection domain, image domain, or using hybrid methods, and enables accurate quantification of contrast agent concentration and organ segmentation. The iodine concentration within the target organ is determined in units such as milligrams per milliliter (mg/mL) and integrated across the segmented organ volume to estimate the total delivered iodine mass, which serves as a surrogate for blood volume (AV). To determine the transit time of the contrast bolus (AT), a time-attenuation curve is generated from real-time bolus tracking images captured at a major arterial site, such as the aorta. This arterial input function (AIF) is fitted with a curve fitting function (e.g., a gamma variate function) to model the time course of iodine arrival, peak, and washout. From the fitted curve, a representative iodine concentration over time duration (g/mL) is extracted. The dual energy image is used to calculate the total iodine mass (g) in the whole-organ tissue compartment. The input iodine concentration and the calculated total iodine mass (g) in the tissue compartment are used to calculate the change in volume of iodinated blood (AV) entering the tissue compartment within the known time interval (AT). The blood flow rate (Q) is then calculated by dividing the estimated iodinated blood volume ΔV by the transit time ΔT, using the equation Q=ΔV/AT. This value is further normalized by the mass or volume of the segmented organ to yield a perfusion value in milliliters per minute per gram of tissue (mL/min/g). The tissue mass may be estimated from the CT data using the number of voxels in the segmented organ. This method enables accurate, quantitative assessment of blood flow that is both physiologically meaningful and standardized across patient populations, while avoiding the motion artifacts and equipment constraints associated with traditional CT perfusion imaging.

In dual-energy computed tomography (CT), material decomposition techniques are employed to distinguish and quantify specific substances within the body by analyzing how different materials attenuate X-rays at two distinct energy levels. When CT data is acquired at a low-energy setting (e.g., 80-100 kVp) and a high-energy setting (e.g., 120-140 kVp), each material exhibits a unique energy-dependent attenuation profile, governed by its atomic number and electron density. The attenuation coefficient μ(E) at energy E can be expressed as a linear combination of attenuation coefficients of two or more basis materials. For example, a voxel's attenuation may be modeled as:

$$μ ( E ) = {w}_{1} \cdot {μ}_{1} ( E ) + {w}_{2} \cdot {μ}_{2} ( E )$$

where μ₁(E) and μ₂(E) are the energy-dependent attenuation coefficients of two known basis materials (e.g., iodine and soft tissue, or calcium and water), and w₁ and w₂ are their respective weighting factors or volume fractions. The basis pair is selected depending on the diagnostic objective—iodine and soft tissue are frequently used in contrast-enhanced perfusion imaging. Using this model, material-specific images can be generated via numerical inversion of the system of equations derived from the attenuation data at both energy levels. These include iodine maps, which display the concentration and spatial distribution of iodine-based contrast agents within the body; virtual non-contrast (VNC) images, in which the iodine contribution is mathematically subtracted to simulate pre-contrast conditions; and effective atomic number (Z-effective) maps and electron density maps, useful in tissue characterization and radiation therapy planning. To perform material decomposition, several algorithms may be used, including Image-domain decomposition, where reconstructed low- and high-energy images are processed post hoc using voxel-wise subtraction and basis material calibration; projection-domain decomposition, in which raw projection data is processed before image reconstruction. This method generally provides higher accuracy and noise performance; and Hybrid methods, combining image- and projection-domain processing to optimize computational efficiency and spatial resolution. For iodine quantification in particular, the system may apply noise-reduction filters, beam hardening correction, and calibration models based on phantom studies to ensure accurate and reproducible measurements.

In the context of the present invention, material decomposition is used not only to generate iodine concentration maps but also to facilitate automated segmentation of the target organ. This is achieved by thresholding the iodine maps to isolate contrast-enhanced regions and applying region-growing or morphological algorithms to delineate organ boundaries. These segmented regions are then used for quantitative analysis, including calculation of contrast uptake, arterial input function modeling via a curve fitting function (e.g., a gamma variate function), and derivation of organ-specific blood flow rates expressed in mL/min/g.

In another aspect, segmentation can be automated based on dual energy material decomposition. Automated segmentation of the organ of interest is performed using dual-energy CT imaging data through a technique known as material decomposition. This process enables the precise identification and isolation of iodine-enhanced tissues from surrounding anatomical structures, improving both accuracy and efficiency in flow measurement workflows. The segmentation begins with the acquisition of dual-energy CT images, which involve capturing two datasets at distinct X-ray energy levels (e.g., low, and high kVp). Due to differences in how various materials attenuate X-rays at different energies, these dual-energy datasets enable the differentiation of contrast agents such as iodine from soft tissue, calcium, or air. Material decomposition is subsequently applied to the acquired images using voxel-wise computational analysis. This analysis separates image components based on their energy-dependent attenuation profiles, allowing for the creation of material-specific maps. Commonly generated maps include iodine maps, soft tissue maps, and calcium maps. These maps are derived from mathematical equations that model the behavior of different materials under dual-energy conditions. Automated segmentation is then executed using these material maps. For example, iodine maps are used to highlight perfused tissues, enabling the identification of organs that have taken up contrast agent. Soft tissue maps aid in excluding bone and air-filled spaces. Image processing software applies thresholding and region-growing algorithms to automatically delineate contiguous areas of interest, such as a contrast-enhanced organ, while excluding irrelevant structures.

The resulting segmentation defines the boundaries of the target organ with minimal or no manual input. This segmented region may then be used directly for further computational steps, including calculating iodine concentration, identifying the time of maximum contrast enhancement, and estimating organ-specific blood flow rates.

This automated segmentation approach offers several advantages over manual methods. It reduces operator-dependent variability, enhances reproducibility and diagnostic accuracy, and significantly improves processing efficiency. Additionally, by leveraging the material discrimination capabilities inherent to dual-energy CT, the system mitigates motion-related and registration artifacts, which are particularly problematic in dynamic or stress imaging scenarios.

In another aspect, the approach is particularly advantageous in emergency room or stress imaging scenarios, where patients may not be able to hold their breath or remain motionless. The minimal scan time and radiation exposure make this method suitable for vulnerable populations while preserving diagnostic accuracy.

In another aspect, this method offers a platform for whole-organ perfusion imaging. For instance, in the lungs, areas with reduced perfusion (measured in mL/min/g) appear as distinct color-coded regions, facilitating rapid clinical interpretation. Similar applications are foreseen in the heart and brain, where precise perfusion mapping could inform intervention. Unlike traditional static CT that merely depicts areas of hypoattenuation suggestive of ischemia, the present method provides absolute perfusion data that directly corresponds to blood flow, enhancing diagnostic specificity and enabling therapy monitoring.

In another aspect, the invention is implemented through a non-transitory computer-readable medium that stores instructions for a computer to execute, encompassing the operations from image receipt to flow rate calculation and output, thereby facilitating the adoption and implementation of the method in various computational environments. Such a non-transitory computer-readable medium can include instructions encoded thereon for:
optionally controlling the administration of an iodine-based contrast agent to the subject;
acquiring preparatory computed tomography (CT) images of the organ, referred to as preparatory CT images;
monitoring distribution of the iodine-based contrast agent using bolus tracking based on the preparatory CT images;
determining a time of maximum contrast enhancement from the preparatory CT images;
acquiring dual-energy computed tomography (CT) images of the organ at or near the determined time of maximum contrast enhancement, referred to as dual-energy CT images;
calculating iodine concentration over time using a curve fitting function based on bolus tracking and the dual-energy CT images; and
quantifying a blood flow rate in the organ based on the calculated iodine concentration.

The following examples illustrate the various embodiments of the present invention. Those skilled in the art will recognize many variations that are within the spirit of the present invention and scope of the claims.

### Blood Flow Measurement Using CT:

Current static CT perfusion techniques provide only relative blood volume and do not measure absolute blood flow, limiting clinical utility. Dynamic CT perfusion methods measure absolute perfusion (mL/min/g) by repeatedly sampling tissue enhancement over time using multiple scans. However, existing dynamic techniques, such as the maximum slope model, rely on monitoring small tissue volumes through multiple (10-20) volume scans, resulting in underestimation of blood flow due to rapid contrast transit (<1 sec) and associated contrast loss from the measurement compartments. These methods also entail high radiation doses, further restricting clinical adoption.

Recent advances enabling rapid whole-organ CT imaging (under ˜3 seconds) address these limitations. By treating the entire organ as a single perfusion compartment, contrast transit times extend to several seconds, allowing for comprehensive image acquisition without contrast loss. This simplifies absolute blood flow measurement using a first-pass analysis (FPA) model and conservation of contrast mass. Blood flow (Q) is determined by quantifying the volume change of iodinated blood (ΔV) entering the compartment over a known time interval (Δt) as:

$$Q = \frac{1}{{C}_{ave}} {( \frac{Δ V}{Δ t} )}_{ave}$$

and perfusion (P) is calculated as Q normalized by compartment mass (M):

$$P = \frac{Q}{M} = \frac{1}{{MC}_{ave}} {( \frac{Δ V}{Δ t} )}_{ave}$$

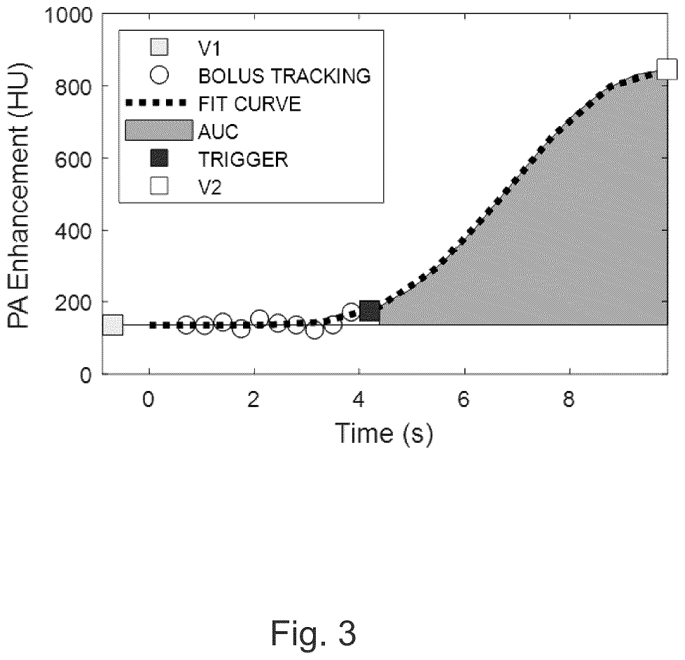

Here, ΔV/Δt represents the iodinated blood volume rate entering the compartment, and Cₐᵥₑ is the average iodine concentration derived from arterial input function (AIF). FIG. 3 illustrates the arterial input function obtained using this method for two-volume CT perfusion. Voxel-by-voxel perfusion (Px,y,z) is further determined by normalizing local enhancement changes (ΔHUx,y,z) against compartment-wide enhancement (ΔHU):

$${P}_{x , y , z} = P \frac{Δ {HU}_{x , y , z}}{Δ HU}$$

Conventionally, accurately obtaining these parameters necessitates multiple scans and high radiation exposure. The described technique reduces radiation exposure by using bolus tracking and a single dual-energy CT angiogram. A gamma variate curve fit of pulmonary artery enhancement from bolus tracking data and the CT angiogram provides the necessary iodine concentration (C_ave).

To eliminate motion misregistration artifacts caused by pre-contrast scans, dual-energy CT angiography employing multi-material decomposition generates iodine concentration maps (mg/cm³). This technique directly estimates ΔV/Δt and C_ave from iodine concentration maps, removing the need for separate non-contrast imaging and significantly reducing radiation dose and measurement error.

In summary, this new dynamic dual-energy CT perfusion technique, enabled by improved temporal resolution, accurately measures absolute blood flow while substantially lowering radiation exposure. It also facilitates concurrent anatomical and physiological assessments from a single contrast injection.

### Validation of the Two-Volume CT Blood Flow Measurement:

The two-volume CT perfusion technique was validated in six swine (mean weight 41.7±10.2 kg), yielding a total of 39 perfusion measurements. Fluorescent microsphere analysis served as the reference standard for quantifying pulmonary blood flow. Different perfusion conditions were induced in each animal through balloon occlusions placed from distal to proximal locations within the pulmonary artery. For each occlusion, more than 20 contrast-enhanced CT images were acquired using a scanning protocol of 320×0.5 mm collimation, 100 kVp, 200 mA, and a 350 ms gantry rotation time. The two-volume CT perfusion method employed retrospective selection of pre-contrast and peak-enhancement volume scans to calculate perfusion. The resulting CT perfusion measurements (P_{CT}) demonstrated a strong correlation with microsphere-derived perfusion values (P_{MIC}), following the regression equation P_{CT}=0.79P_{MIC}+1.32 (r=0.90). Representative CT perfusion maps under various balloon occlusion conditions were constructed. The technique yielded a root-mean-square error (RMSE) of 1.72 mL/min/g and a root-mean-square deviation (RMSD) of 1.50 mL/min/g, with a CT dose index of 8.4 mGy. These findings indicate that the two-volume CT perfusion technique can accurately quantify regional pulmonary perfusion while significantly reducing radiation exposure compared to traditional dynamic CT perfusion approaches. As such, this validated method provides an important foundation for the dual-energy CT perfusion techniques disclosed in the present invention. A more detailed description of this study has been reported elsewhere.

### Estimation of Time to Peak Enhancement in Swine:

An experiment involving 24 swine (mean weight 48.5±14.3 kg) was conducted to determine optimal timing for peak contrast enhancement using dynamic contrast-enhanced CT scans. Enhancement curves of the pulmonary artery were captured over intervals of 20-30 seconds. From these curves, a simulation protocol was developed, whereby one CT scan was performed at baseline aortic enhancement and another at peak enhancement. Across diverse physiological conditions—including varying heart rates, cardiac outputs (1.4-5.1 L/min), injection volumes, and injection durations—the optimal timing to achieve peak pulmonary artery enhancement was consistently predicted as half of the contrast injection duration plus a fixed dispersion delay of approximately 1.0 second. The relationship between pulmonary artery time-to-peak enhancement

$$( \frac{{T}_{Inj}}{2} )$$

and half of the contrast injection duration

$$( ( \frac{{T}_{Inj}}{2} )$$

was strongly linear, described by the equation

$${T}_{PA} = 1.01 \frac{{T}_{Inj}}{2} + 1.01 ( r = 0.95 ) \cdot ) .$$

This accurate timing prediction method is directly relevant to the present invention, ensuring that dual-energy CT acquisitions occur at the precise moment of peak iodine contrast enhancement, thereby significantly enhancing the accuracy and reliability of blood flow measurements. A detailed description of this study is reported elsewhere.

### Vessel-Specific Perfusion Bed Assignment:

A study was conducted to validate an automated minimum-cost path (MCP) assignment technique capable of quantifying lung tissue at risk distal to pulmonary embolism (PE) using only CT angiography. In seven swine (mean weight 42.6±9.6 kg), a total of 33 embolic conditions were created by introducing PE at varying pulmonary artery locations via balloon occlusion under fluoroscopic guidance. Each PE induction was followed by CT angiography and dynamic CT perfusion imaging using a 320-slice CT scanner.

The MCP technique assumes that lung parenchyma is perfused by its nearest arterial branch. A maximally enhanced CT volume from the dynamic scan series was used as the angiographic dataset. Lung tissue was segmented, and centerlines of the pulmonary arteries were extracted. Using a Fast-Marching algorithm, distance maps were generated from each arterial centerline to assign each lung voxel to its nearest supplying artery, thereby defining perfusion territories. The vessel tree was then divided into proximal and distal segments at the balloon midpoint. Tissue mass for each perfusion territory was calculated based on tissue volume, lung parenchymal density (1.053 g/mL), and non-air fraction.

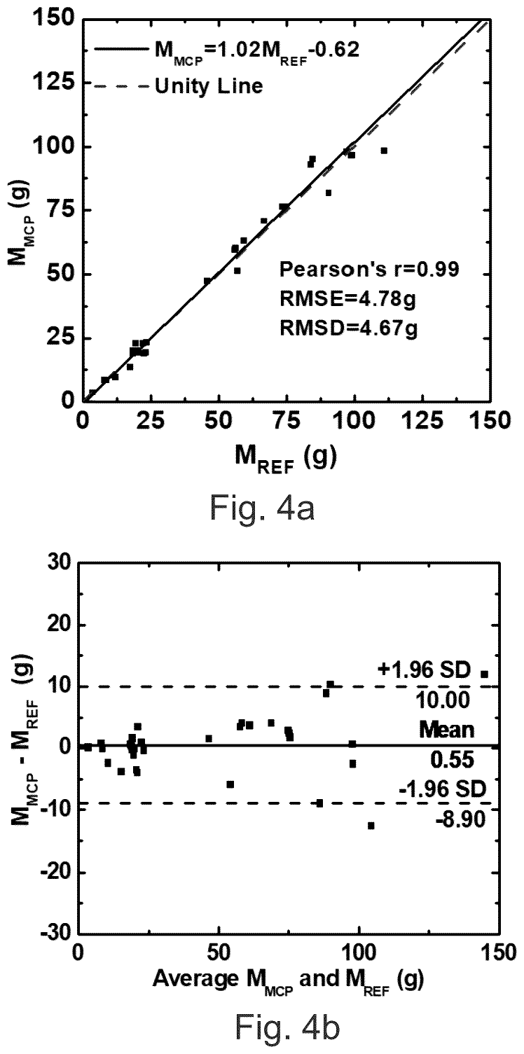

Dynamic CT perfusion served as the reference standard, where regions with perfusion less than 3 mL/min/g were designated as perfusion defects. Maximum intensity projections and perfusion distribution maps, alongside MCP-generated distance maps and segmentations of affected territories were generated. To evaluate the MCP technique's accuracy, lung tissue mass distal to the balloon (M_MCP) was compared to the perfusion defect mass (M_REF) using linear regression and Bland-Altman analysis (FIG. 4). The resulting equation, M_MCP=1.02 M_REF−0.62 g (r=0.99, paired t-test p=0.51), and a Dice similarity coefficient of 0.84+0.08, indicate excellent concordance between anatomical assignments and physiological perfusion deficits.

The reproducibility of the MCP-based assignment technique for lobar segmentation was further evaluated using 46 non-contrast CT scans from 16 Yorkshire swine (mean weight 49.9±4.7 kg). Three independent readers applied a semiautomated algorithm to segment the lungs and extract arterial centerlines, which were subdivided into six arterial subtrees for lobar assignment. Lobar tissue voxels were then assigned to the nearest arterial segment. Linear regression of lobar mass and volume between two acquisitions showed high reproducibility: M_{Lobe1}=0.99 M_{Lobe2}+1.76 (r=0.99, RMSE=7.99 g) and V_{Lobe1}=0.98 V_{Lobe2}+2.66 (r=0.99, RMSE=15.26 mL). These results support the use of the MCP technique for automated lung lobar segmentation, providing a robust framework for regional pulmonary analysis. A more detailed description of this study has been reported elsewhere.

### Quantification of Perfusion Defect:

A study was conducted to determine whether CT perfusion maps can be used to quantify perfusion defects as a percentage of total lung mass. This analysis was based on the MCP validation dataset, in which occlusive pulmonary emboli (PEs) were used. Because the location of the occlusive PEs was known, the assigned perfusion territory distal to each PE—determined using the MCP technique—could be directly compared with the perfusion defect identified on dynamic CT perfusion imaging.

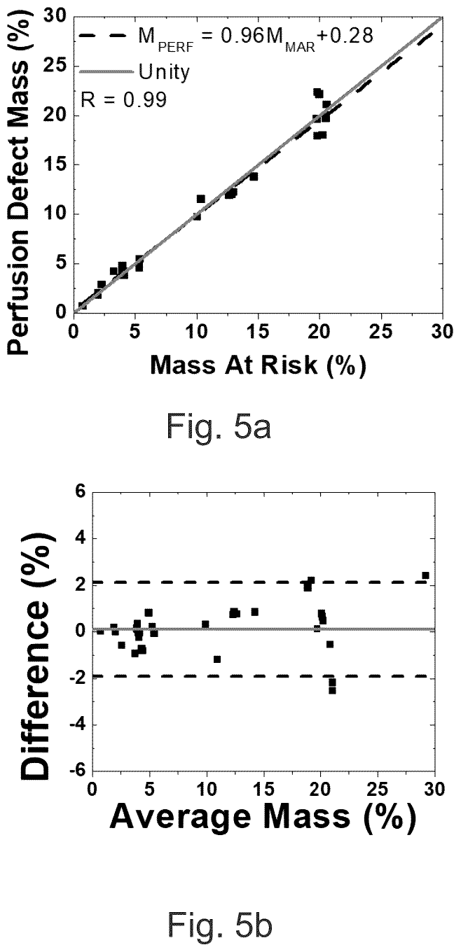

The relationship between perfusion defect mass (M_PERF) and mass at risk (M_MAR) determined via MCP assignment demonstrated strong agreement, as described by the linear regression equation M_PERF=0.98 M_MAR+0.28 (r=0.99). FIG. 5 presents the results of this correlation using linear regression and Bland-Altman analysis. These findings confirm that, for occlusive PEs, anatomical assignment of distal territory closely matches physiologic perfusion defects measured on CT perfusion maps.

This high level of correlation is not necessarily expected in the presence of non-occlusive PEs, where blood flow may still persist in the affected region. In such cases, the anatomical mass at risk may not correspond directly to the physiologically impaired tissue. Accordingly, mass at risk—based on vascular anatomy—and perfusion defect mass—based on blood flow—serve as complementary metrics. Both can be used to assess PE severity, with perfusion defect mass providing a physiologic measurement of acute functional impairment, and mass at risk offering a predictive anatomical assessment when only CT angiography is available.

These metrics are most appropriately expressed in terms of lung mass rather than lung volume, as lung mass remains relatively unaffected by ventilation status. Together, perfusion defect mass and MCP-derived mass at risk represent novel, quantitative parameters for evaluating PE severity and the likelihood of adverse clinical outcomes.

### Multi-Material Decomposition in Dual-Energy CT:

To assess dual-energy CT's capability for precise iodine quantification, a multi-material decomposition algorithm was validated using a dual-energy phantom (Gammex Model 472 Phantom) with calcium (50-400 mg/mL) and iodine inserts (2.0-20 mg/mL). Imaging at 140 kVp produced accurate iodine concentration maps with minimal calcium-related residual errors, confirming reliable iodine quantification. These iodine maps enable voxel-by-voxel iodine concentration measurement (mg/cm³), which is crucial for accurate blood flow assessments and eliminates the necessity for separate non-contrast scans, effectively addressing previous limitations related to motion artifacts.

### Dynamic Dual-Energy CT Perfusion in Patients:

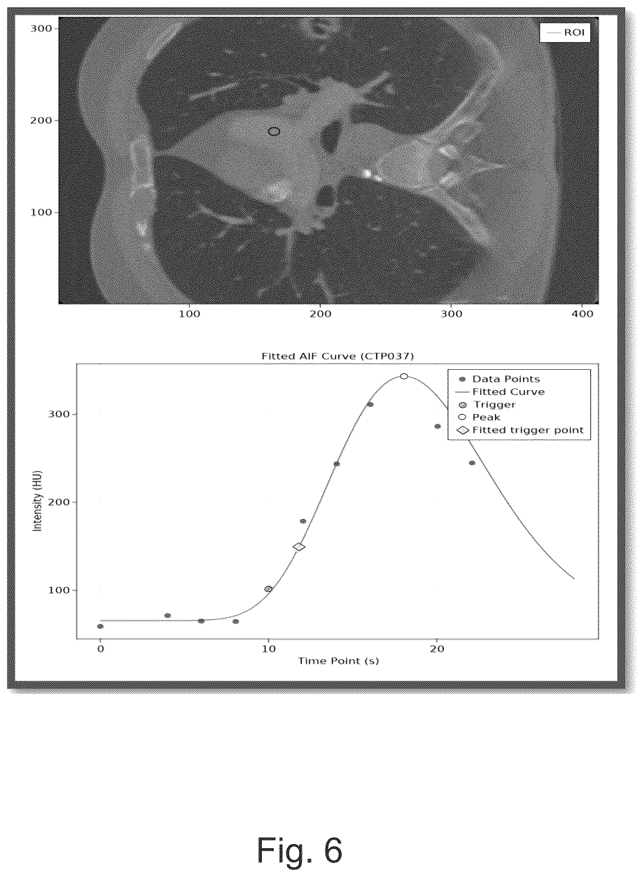

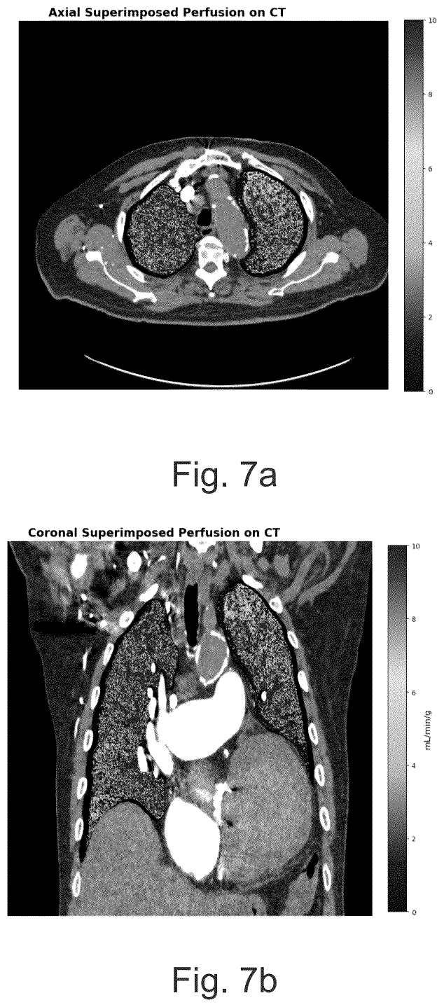

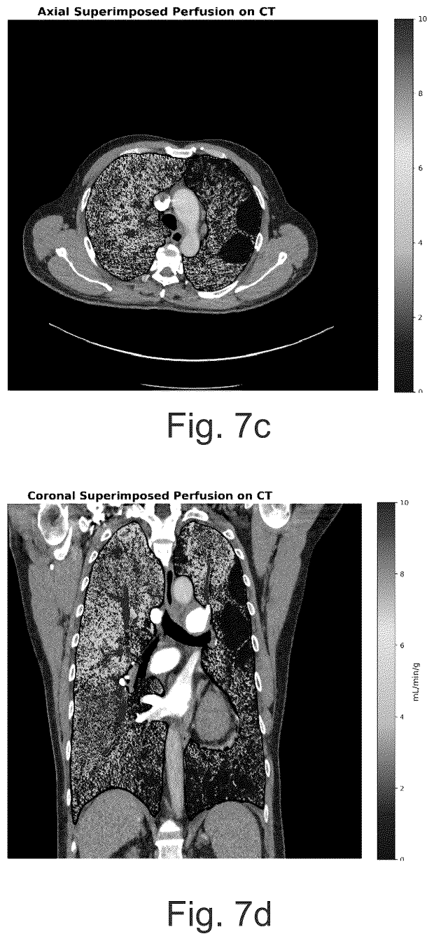

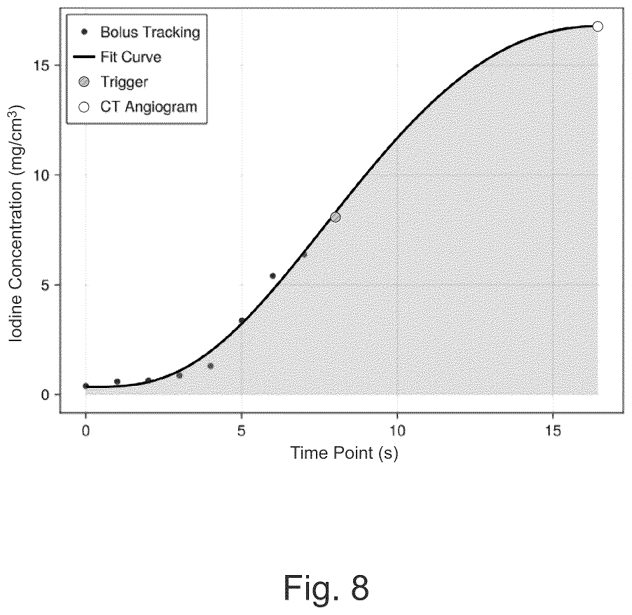

Dynamic dual-energy CT perfusion was implemented clinically using a photon-counting detector CT system (NAEOTOM Alpha, Siemens Medical Systems). Representative flow maps revealed significant differences in blood flow between normal individuals (mean perfusion: 6.54 mL/min/g) and patients with emphysema (mean perfusion: 3.26 mL/min/g), underscoring the technique's clinical applicability in diagnosing microvascular conditions (FIG. 7). The effective radiation dose was notably low (mean: 2.50 mSv). Additionally, patient-specific timing protocols utilizing bolus tracking were validated, accurately predicting peak pulmonary artery enhancement with a mean discrepancy of only 0.15±1.38 seconds, optimizing the timing for dual-energy CT acquisitions (FIGS. 6 and 8).

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. A method for measuring blood flow in an organ of a subject, comprising:
administering an iodine-based contrast agent to the subject;
acquiring preparatory computed tomography (CT) images of the organ, referred to as preparatory CT images;
monitoring distribution of the iodine-based contrast agent using bolus tracking based on the preparatory CT images;
determining a time of maximum contrast enhancement from the preparatory CT images;
acquiring dual-energy computed tomography (CT) images of the organ at or near the determined time of maximum contrast enhancement, referred to as dual-energy CT images;
calculating iodine concentration over time using a curve fitting function based on the bolus tracking and the dual-energy CT images; and
quantifying a blood flow rate in the organ based on the calculated iodine concentration.

2. The method of claim 1, wherein the organ is selected from the group consisting of heart, lungs, brain, kidneys, liver, and lymphatic system.

3. The method of claim 1, further comprising automatically segmenting the organ from surrounding tissues based on dual-energy material decomposition.

4. The method of claim 1, wherein the dual-energy CT images are acquired following standard CT angiography contrast injection protocols.

5. The method of claim 1, wherein the curve fitting function is a gamma variate function.

6. The method of claim 1, wherein the time of maximum contrast enhancement is determined by half of the contrast injection duration and an organ-specific dispersion constant.

7. The method of claim 1, wherein the curve fitting function is applied to a time-intensity curve generated from bolus tracking images acquired at a frame rate from 1 to 5 frames per second.

8. The method of claim 1, wherein calculating the iodine concentration is determined by generating an iodine map from dual-energy CT data using material decomposition.

9. The method of claim 1, wherein quantifying the blood flow rate comprises integrating the iodine concentration over a segmented volume of the organ and dividing by a transit time extracted from the curve fitting function.

10. The method of claim 1, wherein the iodine concentration is calculated in units of milligrams per milliliter and converted to blood volume using a calibration factor based on iodine concentration.

11. The method of claim 1, wherein bolus tracking is performed by monitoring attenuation in an arterial input to detect contrast arrival.

12. A system for measuring blood flow in an organ of a subject, comprising:
a CT scanner capable of dual-energy acquisition configured to acquire images of the organ after administration of contrast agent;
a contrast agent administration unit;
an image processing unit configured to:
determine a time of maximum enhancement of preparatory CT images;
receive dual-energy CT images;
perform automated segmentation of the organ using dual-energy material decomposition;
calculate iodine concentration using a curve fitting function; and
quantify a blood flow rate.

13. The system of claim 12, wherein the organ is selected from the group consisting of heart, lungs, brain, kidneys, liver, and lymphatic system.

14. The system of claim 12, wherein the image processing unit is integrated within the dual-energy CT scanner.

15. The system of claim 12, wherein the image processing unit is configured to generate an arterial input function based on attenuation measurements from bolus tracking images.

16. The system of claim 12, wherein the image processing unit is configured to apply material decomposition configured to generate iodine concentration maps from dual-energy CT data.

17. The system of claim 12, wherein the contrast agent administration unit is an automated injector configured to deliver a bolus of iodine-based contrast followed by a saline flush.

18. The system of claim 12, wherein the image processing unit is further configured to apply a gamma variate curve fit to a time-attenuation profile generated from bolus tracking data.

19. The system of claim 12, wherein the image processing unit includes a segmentation module that identifies organ boundaries by thresholding iodine concentration in material-specific images.

20. The system of claim 12, wherein the dual-energy CT scanner acquires data at two different energy levels.

21. The system of claim 12, wherein the image processing unit is configured to output a perfusion map color-coded by blood flow values in milliliters per minute per gram (mL/min/g).

22. A system for measuring blood flow in an organ of a subject, comprising:
a dual-energy CT scanner configured to acquire images of the organ after administration of an iodine-based contrast agent;
a contrast agent administration unit;
an image processing unit configured to:
optionally control the administration of an iodine-based contrast agent to the subject;
acquire preparatory computed tomography (CT) images of the organ, referred to as preparatory CT images;
monitor distribution of the iodine-based contrast agent using bolus tracking based on the preparatory CT images;
determine a time of maximum contrast enhancement from the preparatory CT images and contrast injection duration;
acquire dual-energy computed tomography (CT) images of the organ at or near the determined time of maximum contrast enhancement, referred to as dual-energy CT images;
calculate iodine concentration over time using a curve fitting function based on the bolus tracking and dual-energy CT images; and
quantify a blood flow rate in the organ based on the calculated iodine concentration.
